SEQUENCE LISTING (8 sequences)

SEQ ID NO: 1 - protein, 22 aa, Zea Mays
  ZM-PDF20 C-terminal propeptide (CTPP)
LAAAEAEADGASQQAVATPRLN

SEQ ID NO: 2 - DNA, 69 bp, Artificial Sequence
  Predicted nucleotide sequence encoding ZM-PDF20 CTPP
ctcgccgccgccgaggctgaggcagatggggcatcgcagcaggccgtggcgacaccgcggctgaactga

SEQ ID NO: 3 - DNA, 1986 bp, Zea Mays
  ZM-UBI Promoter
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgca

SEQ ID NO: 4 - DNA, 72 bp, Artificial Sequence
  cDNA encoding barley alpha amylase (BAA) signal peptide
atggcgaacaagcacctgtccctctccctcttcctcgtgctcctcggcctctccgcctccctcgcctccgga

SEQ ID NO: 5 - protein, 24 aa, Artificial Sequence
  Barley alpha amylase (BAA) signal peptide
MANKHLSLSLFLVLLGLSASLASG

SEQ ID NO: 6 - protein, 4 aa, Artificial Sequence
  Phaseolin C-terminal propeptide (CTPP)
AFVY

SEQ ID NO: 7 - protein, 7 aa, Artificial Sequence
  Tobacco chitinase C-terminal propeptide (CTPP)
GLLVDTM

SEQ ID NO: 8 - DNA, 507 bp, Zea Mays
  CDS (nt 27-341)
cgcgtaccactcctcttcatttcaccatgtcgttcagcaagcgagttgctgtggccgccgccctcgccttggccctgcagcttatcctggcttcacatcttgcagcagctggagccaaaaaagacgaaacaatagcaccgttacagacgtgcgagaccatgatcaaggtcccctgctggggcaaaaacgcgatctgcatccccatctgcatcgcgatgcgctacaccggcggtttctgcgacgtcgccacctgcatgtgcaccaagcaatgcctcgccgccgccgaggctgaggcagatggggcatcgcagcaggccgtggcgacaccgcggctgaactgatggattgtttggtgggcgggcggctggggcatgcatgccgagaataaactcgatgttataacaatgcgaccgagagtagacccaggttgcttgtcatatgtagcagattattgatgaagttatttgatgattgatcgtctcgatctttaacaaaaaaaaaaaaaaa

(19) United States
(12) Patent Application Publication
(10) Pub. No.: US 2012/0054912 A1
(43) Pub. Date: Mar. 1, 2012

(54) Vacuole Targeting Peptides and Methods of Use

(75) Inventors: Virginia C. Crane, Des Moines, IA (US); Nasser Yalpani, Johnston, IA (US); Jeanne M. Sandahl, West Des Moines, IA (US)

(73) Assignee: PIONEER HI-BRED INTERNATIONAL, INC., Johnston, IA (US)

(21) Appl. No.: 13/220,743

(22) Filed: Aug. 30, 2011

Related U.S. Application Data
(60) Provisional application No. 61/379,227, filed on Sep. 1, 2010.

(51) Int. Cl. A01H 5/00; C07K 14/415; A01P 1/00; A01H 5/10; A01N 37/18; C12N 15/29; C12N 15/82

(52) U.S. Cl. 800/279; 536/23.6; 530/326; 435/320.1; 800/298; 800/320.1; 800/320.3; 800/320.2; 800/320; 800/312; 800/306; 800/322; 800/314; 514/2.3; 435/468

(57) ABSTRACT

Compositions and methods for targeting polypeptides to plant vacuoles are provided. Nucleic acid molecules having nucleotide sequences encoding a vacuole-targeting peptide, variants, or fragments thereof are provided. The sequences also can be used for targeting defensin proteins or other polypeptides to vacuoles in plants. Transformed plants, plant cells, tissues, and seed also are provided.

### CROSS-REFERENCE TO RELATED APPLICATIONS

This application claims priority to U.S. Provisional Application No. 61/379,227, filed Sep. 1, 2010, which is hereby incorporated herein in its entirety by reference.

### FIELD OF INVENTION

The invention relates to the field of the genetic manipulation of plants, particularly the targeting of polypeptides of interest to vacuoles in a plant.

### BACKGROUND OF THE INVENTION

The vacuoles of plant cells are multifunctional organelles that are central to the cellular strategies of plant development. The plant vacuole plays various roles that are important in the maintenance of cell organization and function. Signal peptides are used to transport a protein to the plant vacuole. The targeting signal is cleaved off from the precursor protein after their topogenic function is accomplished. Vacuolar sorting signals in yeast vacuolar peoteins are contained within the N-terminal propeptides of the precursor that are cleaved off by vacuolar proteases. Many plant vacuolar proteins contain propeptides that are cleaved off during or after transport to the vacuole.

The propeptides at the C-terminus of some of the vacuolar proteins also contain the vacuolar sorting signals. With prolectin, the vacuolar sorting signal is cleaved off to yield the mature lectin during transport or after deposition in the vacuole. Other proteins contain targeting information within the mature molecule. In the bean legumin, two segments of mature protein were shown to be effective in targeting to the vacuole. These segments include an N-terminal segment of 281 amino acids and a C-terminal segment of 76 amino acids. In bean phytohemagglutinin an internal segment was both sufficient and necessary for directing yeast invertase to vacuoles in transgenic plants.

Disease in plants is caused by biotic and abiotic causes. Biotic causes include fungi, viruses, bacteria, and nematodes. An example of the importance of plant disease is illustrated by phytopathogenic fungi, which cause significant annual crop yield losses as well as devastating epidemics. Plant disease outbreaks have resulted in catastrophic crop failures that have triggered famines and caused major social change. All of the approximately 300,000 species of flowering plants are attacked by pathogenic fungi; however, a single plant species can be host to only a few fungal species, and similarly, most fungi usually have a limited host range. Generally, the best strategy for plant disease control is to use resistant cultivars selected or developed by plant breeders for this purpose. However, the potential for serious crop disease epidemics persists today, as evidenced by outbreaks of the Victoria blight of oats and southern corn leaf blight. Molecular methods of crop protection have the potential to implement novel mechanisms for disease resistance and can also be implemented more quickly than traditional breeding methods. Accordingly, molecular methods are needed to supplement traditional breeding methods to protect plants from pathogen attack.

To aid in the control of plant diseases, methods are needed to increase the levels of antipathogenic polypeptides in the plant. Thus, the present invention provides methods to target heterologous polypeptides to storage vacuoles of the plant.

### SUMMARY OF THE INVENTION

Compositions and methods for targeting polypeptide sequences to plant vacuoles are provided. The compositions increase the expression and accumulation of proteins of interest in the vacuoles of plant cells. Compositions of the invention include C-terminal polypeptides, variants and fragments thereof that are capable of targeting polypeptide sequences to plant vacuoles. These vacuole-targeting sequences can be used to target heterologous polypeptides to vacuoles resulting in high levels of accumulation of proteins in vacuoles.

The methods also involve targeting heterologous proteins such as defensins, other anti-pathogenic polypeptides, endotoxins, and other polypeptides of interest to plant vacuoles by using a vacuole-targeting amino acid sequence of the invention. The compositions and methods in one embodiment can be used for enhancing resistance to plant pathogens including fungal pathogens, plant viruses, microorganisms, nematodes, insects, and the like.

Transformed plants, plant cells, and seeds, as well as methods for making such plants, plant cells, and seeds are additionally provided. Such transformed plants, plant cells, and seeds are transformed with an expression construct encoding the vacuole targeting peptide operably linked to a polypeptide of interest. In this manner, the polypeptide of interest is directed to the vacuoles of the plant cells.

The following embodiments are encompassed by the invention:

1. An isolated polypeptide, wherein expression in a plant of a heterologous polypeptide of interest fused to the polypeptide increases accumulation of the heterologous polypeptide of interest in the plant or targets the heterologous polypeptide of interest to a vacuole in the plant, wherein the polypeptide is selected from the group consisting of:
a) a polypeptide comprising the amino acid sequence set forth in SEQ ID NO:1;
b) a polypeptide having at least 90% sequence identity to SEQ ID NO:1;
c) a polypeptide having at least 95% sequence identity to SEQ ID NO:1; and
d) a polypeptide having at least 15 consecutive amino acids of SEQ ID NO:1.

2. An isolated nucleic acid molecule comprising a nucleotide sequence selected from the group consisting of:
a) a polynucleotide comprising the sequence set forth in SEQ ID NO:2;
b) a polynucleotide having at least 90% sequence identity to SEQ ID NO:2, wherein the polynucleotide encodes a polypeptide, wherein expression in a plant of a heterologous polypeptide of interest fused to the polypeptide increases accumulation of the heterologous polypeptide of interest in the plant or targets the heterologous polypeptide of interest to a vacuole in the plant;
c) a polynucleotide having at least 95% sequence identity to SEQ ID NO:2, wherein the polynucleotide encodes a peptide, wherein expression in a plant of a heterologous polypeptide of interest fused to the polypeptide increases accumulation of the heterologous polypeptide of interest in the plant or targets the heterologous polypeptide of interest to a vacuole in the plant;
d) a polynucleotide encoding the amino acid sequence of SEQ ID NO:1;
e) a polynucleotide encoding an amino acid sequence of a peptide having at least 90% sequence identity to SEQ ID NO:1, wherein expression in a plant of a heterologous polypeptide of interest fused to the polypeptide increases accumulation of the heterologous polypeptide of interest in the plant or targets the heterologous polypeptide of interest to a vacuole in the plant; and
f) a polynucleotide encoding an amino acid sequence of a polypeptide having at least 95% sequence identity to SEQ ID NO:1, wherein expression in a plant of a heterologous polypeptide of interest fused to the polypeptide increases accumulation of the heterologous polypeptide of interest in the plant or targets the heterologous polypeptide of interest to a vacuole in the plant.

3. The nucleic acid molecule of embodiment 2, wherein the nucleotide sequence is optimized for expression in a plant.

4. An expression cassette comprising a promoter that drives expression in a plant or plant cell operably linked to a polynucleotide that encodes a heterologous polypeptide of interest operably linked to a nucleic acid molecule of embodiment 2.

5. The expression cassette of embodiment 4, wherein the heterologous polypeptide of interest is an antifungal polypeptide.

6. The expression cassette of embodiment 4, further comprising an operably linked polynucleotide encoding a signal peptide.

7. The expression cassette of embodiment 6, wherein the polynucleotide encoding the signal peptide comprises the nucleotide sequence of SEQ ID NO:4

8. The expression cassette of embodiment 6, wherein the signal peptide comprises the amino acid sequence of SEQ ID NO:5.

9. A transformed plant cell comprising an expression cassette comprising a promoter that drives expression in a plant cell operably linked to a polynucleotide that encodes a heterologous polypeptide of interest operably linked to a nucleic acid molecule selected from the group consisting of:
a) a polynucleotide comprising the sequence set forth in SEQ ID NO:2;
b) a polynucleotide having at least 90% sequence identity to SEQ ID NO:2, wherein the polynucleotide encodes a polypeptide, wherein expression in a plant of a heterologous polypeptide of interest fused to the polypeptide increases accumulation of the heterologous polypeptide of interest in the plant or targets the heterologous polypeptide of interest to a vacuole in the plant;
c) a polynucleotide having at least 95% sequence identity to SEQ ID NO:2, wherein the polynucleotide encodes a peptide, wherein expression in a plant of a heterologous polypeptide of interest fused to the polypeptide increases accumulation of the heterologous polypeptide of interest in the plant or targets the heterologous polypeptide of interest to a vacuole in the plant;
d) a polynucleotide encoding the amino acid sequence of SEQ ID NO:1;
e) a polynucleotide encoding an amino acid sequence of a peptide having at least 90% sequence identity to SEQ ID NO:1, wherein expression in a plant of a heterologous polypeptide of interest fused to the polypeptide increases accumulation of the heterologous polypeptide of interest in the plant or targets the heterologous polypeptide of interest to a vacuole in the plant; and
f) a polynucleotide encoding an amino acid sequence of a polypeptide having at least 95% sequence identity to SEQ ID NO:1, wherein expression in a plant of a heterologous polypeptide of interest fused to the polypeptide increases accumulation of the heterologous polypeptide of interest in the plant or targets the heterologous polypeptide of interest to a vacuole in the plant.

10. The plant cell of embodiment 9, wherein the heterologous polypeptide of interest is an antifungal polypeptide.

11. The plant cell of embodiment 9, wherein the plant cell displays increased accumulation of the heterologous protein of interest.

12. The plant cell of embodiment 9, wherein the plant cell displays targeting of the heterologous protein of interest to a plant cell vacuole.

13. The plant cell of embodiment 9, wherein the plant cell is from a monocot.

14. The plant cell of embodiment 13, wherein the monocot is maize, wheat, rice, barley, sorghum, or rye.

15. The plant cell of embodiment 9, wherein the plant cell is from a dicot.

16. The plant cell of embodiment 15, wherein the dicot is soybean, Brassica, sunflower, cotton, or alfalfa.

17. A transformed plant comprising an expression cassette comprising a promoter that drives expression in a plant cell operably linked to a polynucleotide that encodes a heterologous polypeptide of interest operably linked to a nucleic acid molecule selected from the group consisting of:
a) a polynucleotide comprising the sequence set forth in SEQ ID NO:2;
b) a polynucleotide having at least 90% sequence identity to SEQ ID NO:2, wherein the polynucleotide encodes a polypeptide, wherein expression in a plant of a heterologous polypeptide of interest fused to the polypeptide increases accumulation of the heterologous polypeptide of interest in the plant or targets the heterologous polypeptide of interest to a vacuole in the plant;
c) a polynucleotide having at least 95% sequence identity to SEQ ID NO:2, wherein the polynucleotide encodes a peptide, wherein expression in a plant of a heterologous polypeptide of interest fused to the polypeptide increases accumulation of the heterologous polypeptide of interest in the plant or targets the heterologous polypeptide of interest to a vacuole in the plant;
d) a polynucleotide encoding the amino acid sequence of SEQ ID NO:1;
e) a polynucleotide encoding an amino acid sequence of a peptide having at least 90% sequence identity to SEQ ID NO:1, wherein expression in a plant of a heterologous polypeptide of interest fused to the polypeptide increases accumulation of the heterologous polypeptide of interest in the plant or targets the heterologous polypeptide of interest to a vacuole in the plant; and
f) a polynucleotide encoding an amino acid sequence of a polypeptide having at least 95% sequence identity to SEQ ID NO:1, wherein expression in a plant of a heterologous polypeptide of interest fused to the polypeptide increases accumulation of the heterologous polypeptide of interest in the plant or targets the heterologous polypeptide of interest to a vacuole in the plant.

18. The plant of embodiment 17, wherein the heterologous polypeptide of interest is an antifungal polypeptide.

19. The plant of embodiment 17, wherein the plant displays increased accumulation of the heterologous protein of interest.

20. The plant of embodiment 17, wherein the plant displays targeting of the heterologous protein of interest to a plant cell vacuole.

21. The plant of embodiment 17, wherein the plant is a monocot.

22. The plant of embodiment 21, wherein the monocot is maize, wheat, rice, barley, sorghum, or rye.

23. The plant of embodiment 17, wherein the plant is a dicot.

24. The plant cell of embodiment 23, wherein the dicot is soybean, Brassica, sunflower, cotton, or alfalfa.

25. A transformed seed of the plant of embodiment 17.

26. A method for inducing plant pathogen resistance in a plant, said method comprising introducing into a plant at least one expression cassette according to embodiment 5.

27. An antipathogenic composition comprising a carrier and at least one polypeptide in accordance with embodiment 1.

28. A method for protecting a plant from a plant pathogen comprising applying the composition according to embodiment 21 to the environment of a plant pathogen.

29. A method for increasing the accumulation of a polypeptide of interest in a vacuoles of a plant cell, said method comprising introducing into said plant cell a nucleic acid construct comprising a C-terminal targeting peptide (CTTP) having the amino acid sequence set forth in SEQ ID NO:1 operably linked to a nucleotide sequence encoding said polypeptide of interest.

30. The method of embodiment 29, wherein said polypeptide of interest is a defensin.

31. The method of embodiment 29, wherein said polypeptide of interest is a delta-endotoxin.

32. The method of embodiment 31, wherein said polypeptide of interest is a cry1Ab toxin.

### DETAILED DESCRIPTION OF THE INVENTION

Overview

The present invention provides compositions and methods for increasing the accumulation of a polypeptide of interest in the vacuoles of a plant, plant cell, or plant part. Compositions of the invention include C-terminal targeting peptides (CTTP) or vacuole-targeting amino acid sequences of defensin proteins. The peptides of the invention target operably linked heterologous polypeptides such as defensins or other anti-pathogenic polypeptides and other polypeptides to plant vacuoles. The plant vacuole serves a storage function. Accordingly, proteins directed to the vacuole remain and accumulate in abundance.

Such vacuole targeting peptides of the invention are C-terminal peptides (CTTP) of plant defensins. Defensins play a role in defense, more specifically plant defense against pathogens, and they share similarity in primary and secondary structure with insect defensins. Plant defensins generally comprise about 45-54 amino acids with four disulfide bridges (Broekaert et al. (1995) Plant Physiol. 108:1353-1358). Defensins inhibit the growth of a broad range of pathogens, including but not limited to fungi, nematocides, bacteria, insects, and viruses at micromolar concentrations. Thus, by “defensin-like activity” it is intended that the peptides inhibit pathogen growth or damage caused by a variety of pathogens, including but not limited to, fungi, insects, nematodes, viruses and bacteria. Defensins inhibit pathogen damage through a variety of mechanisms including, but not limited to, alteration of membrane ion permeability and induction of hyphal branching in fungal targets (Garcia-Olmeda et al. (1998) Biopolymers, Peptide Science 47:479-491, herein incorporated by reference). See, for example, U.S. Pat. No. 6,911,577, herein incorporated by reference.

The vacuole targeting peptides of the invention find use in targeting or directing an operably linked polypeptide or protein from the cytoplasm to the vacuole of the plant cell. The vacuole targeting peptide may comprise from about 15 to about 30 amino acids, including about 18, about 20, about 21, about 22, about 23, about 24, about 25, about 26, about 27, about 28, about 29, up to about 30 amino acids. Vacuole targeting peptides of the invention include the sequence set forth in SEQ ID NO:1 and fragments and variants thereof. Typically after the vacuole targeting peptides have directed a heterologous peptide that is operably linked, they are removed from the targeted protein. In some instances about 1 to about 3 amino acids of the N-terminus of the CTTP can be found at the C′ end of the targeted protein.

The CTTP sequences of the invention can be used to target any polypeptide of interest to the plant vacuole. Polypeptides of interest include those for insect resistance, disease resistance, herbicide resistance, and commercial products. Genes of interest include, generally, those involved in production and accumulation of oil, starch, and carbohydrates. Insect resistant polypeptides include endotoxins with activity towards lepidopteran, dipteran or coleopteran insects as well as polypeptides active against Hymenoptera, Homoptera, Orthoptera and Mallophaga insect orders and to other non-insect organisms like nematodes, mites and protozoa. Such polypeptides include the endotoxins cry1Aa, cry1Ab, cry1Ac, cry1B, cry1C, and the endotoxins listed at the world wide web at lifesci.sussex.ac.uk/home/Neil_Crickmore/Bt/toxins2.html, herein incorporated by reference.

The CTTP sequences of the invention find use in targeting antipathogenic polypeptides to the vacuole. Antipathogenic polypeptides include defensins, thionins, protease inhibitors, amylase inhibitors, scorpion toxin-like proteins, and small cysteine-rich peptides. Defensins are classified in the superfamily of Scorpion toxin-like proteins and in the Plant Defensin family. While not bound by any mechanism of action, expression of the sequences and related genes around disease induced lesions may control symptom development, as in a hypersensitive response (HR), by controlling the protease-mediated cell death mechanism. The defensins also may function directly as antipathogenic proteins by inhibiting proteases produced by pathogens or by binding cell wall components of pathogens. Likewise, they also may act as amphipathic proteins that perturb membrane function, leading to cellular toxicity of the pathogens. The defensins are generally small cysteine-rich peptides and demonstrate antimicrobial activity. By “antimicrobial” or “antimicrobial activity” antibacterial, antiviral, nematocidal, insecticidal, or and antifungal activity is intended. Accordingly, the polypeptides of the invention may enhance resistance to insects and nematodes by increasing accumulation of the antipathogenic polypeptide in plants.

Plant defensins can be grouped into two classes based on precursor structure. Proteins of both classes are targeted to the secretory pathway by an ER signal peptide at their amino-termini. The first, and larger, class of defensins contains no additional targeting information, and thus are secreted to the apoplast. The second class, however, contains a carboxy-terminal propeptide (CTTP). At least one case of dual targeting has been reported for a member of the second class of defensins, in which the mature peptide was identified in both vacuoles (e.g., protein storage vacuoles) and the apoplast, presumably due to removal of the CTTP from a subset of propeptide while moving through the Golgi network.

Mature defensins are characteristically positively charged. As such, the net negative charge of the CTTPs typically matches or is close to the net positive charge of the portion of the precursor destined to form the mature peptide. Thus, other CTTPs can be isolated from this class of proteins and serve as a source of new targeting sequences for heterologous expression of transgenes in plants.

The CTTPs of the invention can be used to target other antipathogenic polypeptides to a plant vacuole. Other antipathogenic polypeptides include proteins, peptides, and lysozymes that naturally occur in insects (Jaynes et al. (1987) Bioassays 6:263-270), plants (Broekaert et al. (1997) Critical Reviews in Plant Sciences 16:297-323), animals (Vunnam et al. (1997) J. Peptide Res. 49:59-66), and humans (Mitra and Zang (1994) Plant Physiol. 106:977-981; Nakajima et al. (1997) Plant Cell Reports 16:674-679) to increase plant disease resistance. Examples of such plant resistance-conferring sequences include those encoding sunflower rhoGTPase-Activating Protein (rhoGAP), lipoxygenase (LOX), Alcohol Dehydrogenase (ADH), and Sclerotinia-Inducible Protein-1 (SCIP-1) described in U.S. Pat. No. 6,709,865, incorporated herein by reference as if set forth in its entirety. These nucleotide sequences enhance plant disease resistance through the modulation of development, developmental pathways, and the plant pathogen defense system. Other plant defense proteins include those described in Int'l Patent Application Publication Nos. WO 99/43823 and WO 99/43821, each of which is incorporated herein by reference as if set forth in its entirety. Additionally, other anti-pathogenic nucleic acid molecules and polypeptides include those described, for example, in Intl Patent Application Publication No. WO 2005/118628; US Patent Application Publication No. 2009/0260106; and U.S. Pat. Nos. 6,121,436; 6,916,970; 7,306,946; 7,589,176 and 7,598,346.

The constructs of the invention can be used in a variety of methods whereby the protein products can be expressed in crop plants and directed to the vacuoles to function as antimicrobial proteins. Expression will result in alterations or modulation of the level, tissue, or timing of expression to achieve enhanced disease, insect, nematode, viral, fungal, or stress resistance. The compositions of the invention may be expressed in the native species including, but not limited to, Arachis hypogaea, Vitis vinifera, Licania michauxii, Cyamopsis tetragonoloba, Parthenium argentatum, Nicotiana benthamiana, Eucalyptus grandis, Tropaeolum majus, Ricinus communis, Vernonia mespilifolia, Chrysobalanus icaco, Glycine max, Triticum aestivum, Oryza sativa, Zea mays, Brassica napus, Tulipa gesneriana, Beta vulgaris, Allium porrum, Amaranthus retroflexus, Hedera helix, Picramnia pentandra, Taraxacum kok-saghyz., Tulipa fosteriana, Momordica charantia, or alternatively, can be heterologously expressed in any plant of interest.

In this manner, the coding sequence for the CTTP can be operably linked to the coding sequence for a heterologous polypeptide and used in combination with a promoter that is introduced into a crop plant. In one embodiment, a high-level expressing constitutive promoter may be utilized and would result in high levels of expression of the heterologous protein.

When the heterologous polypeptide is an antipathogenic polypeptide and more specifically a defensin, the constructs find use in enhancing the plant pathogen defense system. Thus, the compositions and methods of the invention can be used for enhancing resistance to plant pathogens including fungal pathogens, plant viruses, insect pathogens, bacterial pathogens, nematodes, and the like. In this manner, the method involves stably transforming a plant with a nucleotide sequence capable of modulating the plant pathogen defense system operably linked to a CTTP and such construct operably linked with a promoter capable of driving expression of a gene in a plant cell. By “enhancing resistance” increasing the tolerance of the plant to pathogens is intended. That is, the defensin may slow or prevent pathogen infection and/or spread.

The article “a” and “an” are used herein to refer to one or more than one (i.e., to at least one). By way of example, “an element” means one or more element.

As used herein, the term “plant” includes a whole plant as well as plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

Compositions

The nucleotide sequences of the invention are sequences comprising nucleic acid molecules that encode a vacuole-targeting peptide and variants and fragments thereof. In particular, nucleotide sequences include sequences that encode the amino acid sequence set forth in SEQ ID NO:1, and the nucleotide sequence set forth in SEQ ID NO:2.

The invention therefore encompasses isolated or substantially purified nucleic acid molecules or proteins. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid molecule is free of sequences (preferably protein encoding sequences) that naturally flank it (i.e., sequences located at the 5′ and 3′ ends of the nucleic acid) in the genomic DNA of the organism from which it is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank it in genomic DNA of the cell from which it is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, preferably culture medium represents less than about 30%, 20%, 10%, 5%, or 1% (by dry weight) of chemical precursors or non-protein-of-interest chemicals.

Fragments and variants of the disclosed nucleic acid molecules and proteins encoded thereby also are encompassed by the present invention. By “fragment” is intended a portion of the nucleotide sequence of the nucleic acid molecules or a portion of the amino acid sequence and hence protein encoded thereby. Fragments of a nucleotide sequence may encode protein fragments that retain the biological activity of the native protein. Fragments of a CTTP retain the ability to target polypeptides to a vacuole. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence of the nucleic acid molecule encoding the proteins of the invention.

Nucleic acid molecules that are fragments of a CPTT comprise at least about 30, about 33, about 36, about 39, about 45, about 48, about 51, about 54, about 57, about 60, about 63 nucleotides, or up to the total number of nucleotides in a full-length CTTP.

By “variants” substantially similar sequences are intended. For nucleotide sequences of the nucleic acid molecules of the invention, conservative variants include those sequences that, because of the degeneracy of the genetic code, encode the amino acid sequence of one of the defensin polypeptides of the invention. Naturally occurring allelic variants such as these can be identified with the use of well-known molecular biology techniques, such as, for example, with polymerase chain reaction (PCR) and hybridization techniques as outlined below. Variant nucleotide sequences also include synthetically derived nucleotide sequences, such as those generated, for example, by using site-directed mutagenesis but which still encode a CTTP of the invention. Generally, variants of a particular nucleotide sequence of the invention will have at least about 75%, 80%, 85%, preferably at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant protein or polypeptide” a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein is intended. Variant proteins encompassed by the present invention are biologically active, that is, they continue to possess the desired biological activity of the native protein or peptide sequence, such as, for example, the ability to target a polypeptide to a plant vacuole as described herein. Such variants may result from, for example, genetic polymorphism or from human manipulation. Biologically active variants of a native defensin protein of the invention will have at least about 75%, 80%, 85%, preferably at least about 90%, 91%, 92%, 93%, 94%, 95%, 96%, 97%, and more preferably at least about 98%, 99% or more sequence identity to the amino acid sequence for the native CTTP as determined by sequence alignment programs described elsewhere herein using default parameters. Fragments of a CTTP amino acid molecule that retains biological activity comprise at least about 10, about 11, about 12, about 13, about 14, about 15, about 16, about 17, about 18, about 19, about 20 contiguous amino acids up to the full-length peptide of the CTTP.

Biological activity of the CTTP can be arrayed by operably linking the peptide to a heterologous polypeptide and arraying for presence of the heterologous polypeptide in a vacuole. Such methods are known in the art.

The polypeptides of the invention may be altered in various ways including amino acid substitutions, deletions, truncations, and insertions. Novel proteins having properties of interest may be created by combining elements and fragments of proteins of the present invention as well as other proteins. Methods for such manipulations are generally known in the art. For example, amino acid sequence variants can be prepared by mutations in the DNA. Methods for mutagenesis and nucleotide sequence alterations are well known in the art. See, e.g., Kunkel (1985) Proc. Natl. Acad. Sci. USA 82:488-492; Kunkel et al. (1987) Methods in Enzymol. 154:367-382; U.S. Pat. No. 4,873,192; Techniques in Molecular Biology (Walker & Gaastra eds., Macmillan Publishing Co. 1983) and the references cited therein. Guidance as to appropriate amino acid substitutions that do not affect biological activity of the CTTP of interest may be found in the model of Dayhoff et al. Atlas of Protein Sequence and Structure (Natl. Biomed. Res. Found., Washington, D.C. 1978), incorporated herein by reference as if set forth in its entirety. Conservative substitutions, such as exchanging one amino acid with another having similar properties, may be preferred.

Thus, nucleotide sequences of the nucleic acid molecules of the invention include both the naturally occurring sequences as well as mutant forms Likewise, the proteins of the invention encompass naturally occurring proteins as well as variations and modified forms thereof. Such variants will continue to possess the desired developmental activity, or defense response activity. Obviously, the mutations that will be made in the DNA encoding the variant must not place the sequence out of reading frame.

The deletions, insertions, and substitutions of the sequences encompassed herein are not expected to produce radical changes in the characteristics of the targeting peptide. However, when it is difficult to predict the exact effect of the substitution, deletion, or insertion in advance of doing so, one skilled in the art will appreciate that the effect will be evaluated by routine screening assays.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988), supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997), supra. When utilizing BLAST, Gapped BLAST, PSI-BLAST, the default parameters of the respective programs (e.g., BLASTN for nucleotide sequences, BLASTX for proteins) can be used. See on the World Wide Web at ncbi.hlm.nih.gov. Alignment may also be performed manually by inspection.

Unless otherwise stated, sequence identity/similarity values provided herein refer to the value obtained using GAP Version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Disease and Pests

The CTTPs of the invention when operably linked to an antipathogenic polypeptide and expressed in a plant cell are useful in protecting plants against fungal pathogens, viruses, nematodes, insects, and the like. Thus, transformed plants, plant cells, plant tissues and seeds thereof are encompassed by the compositions of the invention.

By “plant pathogen” or “plant pest” any organism that can cause harm to a plant, by inhibiting or slowing the growth of a plant, by damaging the tissues of a plant, by weakening the immune system of a plant, reducing the resistance of a plant to abiotic stresses, and/or by causing the premature death of the plant, etc. is intended. Plant pathogens and plant pests include insects, nematodes, and organisms such as fungi, viruses, and bacteria.

By “disease resistance” or “pathogen resistance” it is intended that the organisms avoid the disease symptoms which are the outcome of organism-pathogen interactions. That is, pathogens are prevented from causing diseases and the associated disease symptoms, or alternatively, the disease symptoms caused by the pathogen is minimized or lessened.

By “anti-pathogenic compositions” is intended that the compositions of the invention are capable of suppressing, controlling, and/or killing the invading pathogenic organism. An antipathogenic composition of the invention will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

An “antimicrobial agent,” a “pesticidal agent,” a “defensin,” an “antiviral agent,” and “insecticidal agent,” and/or a “fungicidal agent” will act similarly to suppress, control, and/or kill the invading pathogen.

A defensive agent will possess defensive activity. By “defensive activity” an antipathogenic, antimicrobial, antiviral, insecticidal, or antifungal activity is intended.

By “antipathogenic compositions” it is intended that the compositions of the invention have activity against pathogens; including fungi, microorganisms, viruses, insects and nematodes, and thus are capable of suppressing, controlling, and/or killing the invading pathogenic organism. An antipathogenic composition of the invention will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect organisms, particularly plants, from disease, particularly those diseases that are caused by invading pathogens.

Assays that measure antipathogenic activity are commonly known in the art, as are methods to quantitate disease resistance in plants following pathogen infection. See, for example, U.S. Pat. No. 5,614,395, incorporated herein by reference as if set forth in its entirety. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, e.g., Thomma et al. (1998) Plant Biology 95:15107-15111, incorporated herein by reference as if set forth in its entirety.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, incorporated herein by reference as if set forth in its entirety). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, each of which is incorporated herein by reference as if set forth in its entirety). The methods of the invention can be used with other methods available in the art for enhancing disease resistance in plants. For example, any one of a variety of second nucleotide sequences may be utilized, embodiments of the invention encompass those second nucleotide sequences that, when expressed in a plant, help to increase the resistance of a plant to pathogens. It is recognized that such second nucleotide sequences may be used in either the sense or antisense orientation depending on the desired outcome. Other plant defense proteins include those described in Int'l Patent Application Publication Nos. WO 99/43823 and WO 99/43821, each of which are incorporated herein by reference as if set forth in its entirety.

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal and viral pathogens for the major crops include: Soybeans: Phytophthora megasperma f.sp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines, Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibacter Michigan's subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium spp., Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Tilletia indica, Pythium gramicola, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum p.v. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydis (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudomonas avenae, Erwinia chrysanthemi p.v. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Periconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, and lesion nematodes, including Heterodera and Globodera spp.; particularly Globodera rostochiensis and Globodera pailida (potato cyst nematodes); Heterodera glycines (soybean cyst nematode); Heterodera schachtii (beet cyst nematode); and Heterodera avenae (cereal cyst nematode). Additional nematodes include: Heterodera cajani; Heterodera trifolii; Heterodera oryzae; Globodera tabacum; Meloidogyne incognita; Meloidogyne javonica; Meloidogyne hapla; Meloidogyne arenaria; Meloidogyne naasi; Meloidogyne exigua; Xiphinema index; Xiphinema italiae; Xiphinema americanum; Xiphinema diversicaudatum; Pratylenchus penetrans; Pratylenchus brachyurus; Pratylenchus zeae; Pratylenchus coffeae; Pratylenchus thornei; Pratylenchus scribneri; Pratylenchus vulnus; Pratylenchus curvitatus; Radopholus similis; Radopholus citrophilus; Ditylenchus dipsaci; Helicotylenchus multicintus; Rotylenchulus reniformis; Belonolaimus spp.; Paratrichodorus anemones; Trichodorus spp.; Primitivus spp.; Anguina tritici; Bider avenae; Subanguina radicicola; Tylenchorhynchus spp.; Haplolaimus seinhorsti; Tylenchulus semipenetrans; Hemicycliophora arenaria; Belonolaimus langicaudatus; Paratrichodorus xiphinema; Paratrichodorus christiei; Rhadinaphelenchus cocophilus; Paratrichodorus minor; Hoplolaimus galeatus; Hoplolaimus columbus; Criconemella spp.; Paratylenchus spp.; Nacoabbus aberrans; Aphelenchoides besseyi; Ditylenchus angustus; Hirchmaniella spp.; Scutellonema spp.; Hemicriconemoides kanayaensis; Tylenchorynchus claytoni; and Cacopaurus pestis.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blotch leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia spp., Root maggots.

As indicated, the targeting peptides of the invention can be used to target insect toxins to a plant vacuole. Thus, they can be used to target polypeptides in plants to control insect pests. Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera.

Insects of the order Lepidoptera include, but are not limited to, armyworms, cutworms, loopers, and heliothines in the family Noctuidae Agrotis ipsilon Hufnagel (black cutworm); A. orthogonia Morrison (western cutworm); A. segetum Denis & Schiffermüller (turnip moth); A. subterranea Fabricius (granulate cutworm); Alabama argillacea Hübner (cotton leaf worm); Anticarsia gemmatalis Hübner (velvetbean caterpillar); Athetis mindara Barnes and McDunnough (rough skinned cutworm); Earias insulana Boisduval (spiny bollworm); E. vittella Fabricius (spotted bollworm); Egira (Xylomyges) curialis Grote (citrus cutworm); Euxoa messoria Harris (darksided cutworm); Helicoverpa armigera Hübner (American bollworm); H. zea Boddie (corn earworm or cotton bollworm); Heliothis virescens Fabricius (tobacco budworm); Hypena scabra Fabricius (green cloverworm); Hyponeuma tabula Schaus; (Mamestra configurata Walker (bertha armyworm); M. brassicae Linnaeus (cabbage moth); Melanchra picta Harris (zebra caterpillar); Mocis latipes Guenée (small mocis moth); Pseudaletia unipuncta Haworth (armyworm); Pseudoplusia includens Walker (soybean looper); Richia albicosta Smith (Western bean cutworm);Spodoptera frugiperda JE Smith (fall armyworm); S. exigua Hübner (beet armyworm); S. litura Fabricius (tobacco cutworm, cluster caterpillar); Trichoplusia ni Hübner (cabbage looper); borers, casebearers, webworms, coneworms, and skeletonizers from the families Pyralidae and Crambidae such as Achroia grisella Fabricius (lesser wax moth); Amyelois transitella Walker (naval orangeworm); Anagasta kuehniella Zeller (Mediterranean flour moth); Cadra cautella Walker (almond moth); Chilo partellus Swinhoe (spotted stalk borer); C. suppressalis Walker (striped stem/rice borer); C. terrenellus Pagenstecher (sugarcane stemp borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hübner (grape leaffolder); Diaphania hyalinata Linnaeus (melon worm); D. nitidalis Stoll (pickleworm); Diatraea flavipennella Box; D. grandiosella Dyar (southwestern corn borer), D. saccharalis Fabricius (surgarcane borer); Elasmopalpus lignosellus Zeller (lesser cornstalk borer); Eoreuma loftini Dyar (Mexican rice borer); Ephestia elutella Hübner (tobacco (cacao) moth); Galleria mellonella Linnaeus (greater wax moth); Hedylepta accepta Butler (sugarcane leafroller); Herpetogramma licarsisalis Walker (sod webworm); Homoeosoma electellum Hulst (sunflower moth); Loxostege sticticalis Linnaeus (beet webworm); Maruca testulalis Geyer (bean pod borer); Orthaga thyrisalis Walker (tea tree web moth); Ostrinia nubilalis Hübner (European corn borer); Plodia interpunctella Hübner (Indian meal moth); Scirpophaga incertulas Walker (yellow stem borer); Udea rubigalis Guenée (celery leaftier); and leafrollers, budworms, seed worms, and fruit worms in the family Tortricidae Acleris gloverana Walsingham (Western blackheaded budworm); A. variana Fernald (Eastern blackheaded budworm); Adoxophyes orana Fischer von Rösslerstamm (summer fruit tortrix moth); Archips spp. including A. argyrospila Walker (fruit tree leaf roller) and A. rosana Linnaeus (European leaf roller); Argyrotaenia spp.; Bonagota salubricola Meyrick (Brazilian apple leafroller); Choristoneura spp.; Cochylis hospes Walsingham (banded sunflower moth); Cydia latiferreana Walsingham (filbertworm); C. pomonella Linnaeus (codling moth); Endopiza viteana Clemens (grape berry moth); Eupoecilia ambiguella Hübner (vine moth); Grapholita molesta Busck (oriental fruit moth); Lobesia botrana Denis & Schiffermüller (European grape vine moth); Platynota flavedana Clemens (variegated leafroller); P. stultana Walsingham (omnivorous leafroller); Spilonota ocellana Denis & Schiffermüller (eyespotted bud moth); and Suleima helianthana Riley (sunflower bud moth).

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Silkmoth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Erechthias flavistriata Walsingham (sugarcane bud moth); Euproctis chrysorrhoea Linnaeus (browntail moth); Harrisina americana Guérin-Méneville (grapeleaf skeletonizer); Heliothis subflexa Guenée; Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Malacosoma spp.; Manduca quinquemaculata Haworth (five spotted hawk moth, tomato hornworm); M. sexta Haworth (tomato hornworm, tobacco hornworm); Operophtera brumata Linnaeus (winter moth); Orgyia spp.; Paleacrita vernata Peck (spring cankerworm); Papilio cresphontes Cramer (giant swallowtail, orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Telchin licus Drury (giant sugarcane borer); Thaumetopoea pityocampa Schiffermüller (pine processionary caterpillar); Tineola bisselliella Hummel (webbing clothesmoth); Tuta absoluta Meyrick (tomato leafminer) and Yponomeuta padella Linnaeus (ermine moth).

Of interest are larvae and adults of the order Coleoptera including weevils from the families Anthribidae, Bruchidae, and Curculionidae including, but not limited to: Anthonomus grandis Boheman (boll weevil); Cylindrocopturus adspersus LeConte (sunflower stem weevil); Diaprepes abbreviatus Linnaeus (Diaprepes root weevil); Hypera punctata Fabricius (clover leaf weevil); Lissorhoptrus oryzophilus Kuschel (rice water weevil); Metamasius hemipterus hemipterus Linnaeus (West Indian cane weevil); M. hemipterus sericeus Olivier (silky cane weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug); S. livis Vaurie (sugarcane weevil); Rhabdoscelus obscurus Boisduval (New Guinea sugarcane weevil); flea beetles, cucumber beetles, rootworms, leaf beetles, potato beetles, and leafminers in the family Chrysomelidae including, but not limited to: Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabaeidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, Eleodes spp., Melanotus spp. including M. communis Gyllenhal (wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

Adults and immatures of the order Diptera are of interest, including leafminers Agromyza parvicornis Loew (corn blotch leafminer); midges including, but not limited to: Contarinia sorghicola Coquillett (sorghum midge); Mayetiola destructor Say (Hessian fly); Neolasioptera murtfeldtiana Felt, (sunflower seed midge); Sitodiplosis mosellana Gélin (wheat midge); fruit flies (Tephritidae), Oscinella frit Linnaeus (frit flies); maggots including, but not limited to: Delia spp. including Delia platura Meigen (seedcorn maggot); D. coarctata Fallen (wheat bulb fly); Fannia canicularis Linnaeus, F. femoralis Stein (lesser house flies); Meromyza americana Fitch (wheat stem maggot); Musca domestica Linnaeus (house flies); Stomoxys calcitrans Linnaeus (stable flies)); face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds); and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are those of the order Hemiptera such as, but not limited to, the following families: Adelgidae, Aleyrodidae, Aphididae, Asterolecamidae, Cercopidae, Cicadellidae, Cicadidae, Cixiidae, Coccidae, Coreidae, Dactylopiidae, Delphacidae, Diaspididae, Eriococcidae, Flatidae, Fulgoridae, Issidae, Lygaeidae, Margarodidae, Membracidae, Miridae, Ortheziidae, Pentatomidae, Phoenicococcidae, Phylloxeridae, Pseudococcidae, Psyllidae, Pyrrhocoridae and Tingidae.

Agronomically important members from the order Hemiptera include, but are not limited to: Acrosternum hilare Say (green stink bug); Acyrthisiphon pisum Harris (pea aphid); Adelges spp. (adelgids); Adelphocoris rapidus Say (rapid plant bug); Anasa tristis De Geer (squash bug); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacaspis tegalensis Zehntner (sugarcane scale); Aulacorthum solani Kaltenbach (foxglove aphid); Bemisia tabaci Gennadius (tobacco whitefly, sweetpotato whitefly); B. argentifolii Bellows & Perring (silverleaf whitefly); Blissus leucopterus leucopterus Say (chinch bug); Blostomatidae spp.; Brevicoryne brassicae Linnaeus (cabbage aphid); Cacopsylla pyricola Foerster (pear psylla); Calocoris norvegicus Gmelin (potato capsid bug); Chaetosiphon fragaefolii Cockerell (strawberry aphid); Cimicidae spp.; Coreidae spp.; Corythuca gossypii Fabricius (cotton lace bug); Cyrtopeltis modesta Distant (tomato bug); C. notatus Distant (suckfly); Deois flavopicta Stål (spittlebug); Dialeurodes citri Ashmead (citrus whitefly); Diaphnocoris chlorionis Say (honeylocust plant bug); Diuraphis noxia Kurdjumov/Mordvilko (Russian wheat aphid); Duplachionaspis divergens Green (armored scale); Dysaphis plantaginea Paaserini (rosy apple aphid); Dysdercus suturellus Herrich-Schäffer (cotton stainer); Dysmicoccus boninsis Kuwana (gray sugarcane mealybug); Empoasca fabae Harris (potato leafhopper); Eriosoma lanigerum Hausmann (woolly apple aphid); Erythroneoura spp. (grape leafhoppers); Eumetopina flavipes Muir (Island sugarcane planthopper); Eurygaster spp.; Euschistus servus Say (brown stink bug); E. variolarius Palisot de Beauvois (one-spotted stink bug); Graptostethus spp. (complex of seed bugs); and Hyalopterus pruni Geoffroy (mealy plum aphid); Icerya purchasi Maskell (cottony cushion scale); Labopidicola allii Knight (onion plant bug); Laodelphax striatellus Fallen (smaller brown planthopper); Leptoglossus corculus Say (leaf-footed pine seed bug); Leptodictya tabida Herrich-Schaeffer (sugarcane lace bug); Lipaphis erysimi Kaltenbach (turnip aphid); Lygocoris pabulinus Linnaeus (common green capsid); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Macrosiphum euphorbiae Thomas (potato aphid); Macrosteles quadrilineatus Forbes (aster leafhopper); Magicicada septendecim Linnaeus (periodical cicada); Mahanarva fimbriolata Stål (sugarcane spittlebug); M. posticata Stål (little cicada of sugarcane); Melanaphis sacchari Zehntner (sugarcane aphid); Melanaspis glomerata Green (black scale); Metopolophium dirhodum Walker (rose grain aphid); Myzus persicae Sulzer (peach-potato aphid, green peach aphid); Nasonovia ribisnigri Mosley (lettuce aphid); Nephotettix cinticeps Uhler (green leafhopper); N. nigropictus Stål (rice leafhopper); Nezara viridula Linnaeus (southern green stink bug); Nilaparvata lugens Stål (brown planthopper); Nysius ericae Schilling (false chinch bug); Nysius raphanus Howard (false chinch bug); Oebalus pugnax Fabricius (rice stink bug); Oncopeltus fasciatus Dallas (large milkweed bug); Orthops campestris Linnaeus; Pemphigus spp. (root aphids and gall aphids); Peregrinus maidis Ashmead (corn planthopper); Perkinsiella saccharicida Kirkaldy (sugarcane delphacid); Phylloxera devastatrix Pergande (pecan phylloxera); Planococcus citri Risso (citrus mealybug); Plesiocoris rugicollis Fallen (apple capsid); Poecilocapsus lineatus Fabricius (four-lined plant bug); Pseudatomoscelis seriatus Reuter (cotton fleahopper); Pseudococcus spp. (other mealybug complex); Pulvinaria elongata Newstead (cottony grass scale); Pyrilla perpusilla Walker (sugarcane leafhopper); Pyrrhocoridae spp.; Quadraspidiotus perniciosus Comstock (San Jose scale); Reduviidae spp.; Rhopalosiphum maidis Fitch (corn leaf aphid); R. padi Linnaeus (bird cherry-oat aphid); Saccharicoccus sacchari Cockerell (pink sugarcane mealybug); Scaptacoris castanea Perty (brown root stink bug); Schizaphis graminum Rondani (greenbug); Sipha flava Forbes (yellow sugarcane aphid); Sitobion avenae Fabricius (English grain aphid); Sogatella furcifera Horvath (white-backed planthopper); Sogatodes oryzicola Muir (rice delphacid); Spanagonicus albofasciatus Reuter (whitemarked fleahopper); Therioaphis maculata Buckton (spotted alfalfa aphid); Tinidae spp.; Toxoptera aurantii Boyer de Fonscolombe (black citrus aphid); and T. citricida Kirkaldy (brown citrus aphid); Trialeurodes abutiloneus (bandedwinged whitefly) and T. vaporariorum Westwood (greenhouse whitefly); Trioza diospyri Ashmead (persimmon psylla); and Typhlocyba pomaria McAtee (white apple leafhopper).

Also included are adults and larvae of the order Acari (mites) such as Aceria tosichella Keifer (wheat curl mite); Panonychus ulmi Koch (European red mite); Petrobia latens Müller (brown wheat mite); Steneotarsonemus bancrofti Michael (sugarcane stalk mite); spider mites and red mites in the family Tetranychidae, Oligonychus grypus Baker & Pritchard, O. indicus Hirst (sugarcane leaf mite), O. pratensis Banks (Banks grass mite), O. stickneyi McGregor (sugarcane spider mite); Tetranychus urticae Koch (two spotted spider mite); T. mcdanieli McGregor (McDaniel mite); T. cinnabarinus Boisduval (carmine spider mite); T. turkestani Ugarov & Nikolski (strawberry spider mite), flat mites in the family Tenuipalpidae, Brevipalpus lewisi McGregor (citrus flat mite); rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick); and scab and itch mites in the families Psoroptidae, Pyemotidae, and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch & Mulaik (brown recluse spider); and the Latrodectus mactans Fabricius (black widow spider); and centipedes in the order Scutigeromorpha such as Scutigera coleoptrata Linnaeus (house centipede). In addition, insect pests of the order Isoptera are of interest, including those of the termitidae family, such as, but not limited to, Cornitermes cumulans Kollar, Cylindrotermes nordenskioeldi Holmgren and Pseudacanthotermes militaris Hagen (sugarcane termite); as well as those in the Rhinotermitidae family including, but not limited to Heterotermes tenuis Hagen. Insects of the order Thysanoptera are also of interest, including but not limited to thrips, such as Stenchaetothrips minutus van Deventer (sugarcane thrips).

Expression Cassettes

The nucleic acid sequences of the present invention can be expressed in a host cell such as bacterial, fungal, yeast, insect, mammalian, or preferably plant cells. It is expected that one of skill in the art is knowledgeable in the numerous expression systems available for expression of a nucleic acid encoding a protein of the present invention. No attempt to describe in detail the various methods known for the expression of proteins in prokaryotes or eukaryotes will be made.

As used herein, “heterologous” in reference to a nucleic acid molecule is a nucleic acid molecule that originates from a foreign species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

The CTTPs of the invention are provided in expression cassettes or DNA constructs for expression in the plant of interest. The cassette will include the CTTP operably linked to a heterologous polypeptide of interest to form a CTTP-heterologous polypeptide construct. Additionally, the cassette will include 5′ and 3′ regulatory sequences operably linked to the CTTP-heterologous polypeptide construct of the invention. By “operably linked” a functional linkage between a first and a second sequence, wherein the first sequence retains function and does not disrupt the coding sequence of the polypeptide. For example, promoter sequences initiate and mediate transcription of the DNA sequence corresponding to the second sequence expressing an operative amino acid sequence. Generally, “operably linked” means that the nucleic acid sequences being linked are contiguous and, where necessary to join two protein coding regions, the regions are contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the sequences to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a polynucleotide encoding a vacuolar targeting peptide, a polypeptide of interest that is heterologous with respect to the vacuolar targeting peptide, and a transcriptional and translational termination region functional in plants.

The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, imidazolinones, and 2,4-dichlorophenoxyacetate (2,4-D).

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 promoter and other constitutive promoters disclosed in Int'l Patent Application Publication No. WO 99/43838 and U.S. Pat. No. 6,072,050; the core CaMV ³⁵S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142; and 6,177,611, each of which is incorporated herein by reference as if set forth its entirety.

Inducible promoters may be utilized, particularly a pathogen-inducible promoter. Such promoters include those from pathogenesis-related proteins (PR proteins), which are induced following infection by a pathogen; for example, PR proteins, SAR proteins, beta-1,3-glucanase, chitinase, etc. See, e.g., Redolfi et al. (1983) Neth. J. Plant Pathol. 89:245-254; Uknes et al. (1992) Plant Cell 4:645-656; and Van Loon (1985) Plant Mol. Virol. 4:111-116. See also Int'l Patent Application Publication No. WO 99/43819, incorporated herein by reference as if set forth in its entirety.

Of interest are promoters that are expressed locally at or near the site of pathogen infection. See, e.g., Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, e.g., Cordero et al. (1992) Physiol. Mol. Plant. Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2 (U.S. Pat. No. 5,428,148); win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, each of which is incorporated herein by reference as if set forth in its entirety.

Leaf-specific promoters are known in the art. See, e.g., Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Transformation protocols as well as protocols for introducing nucleotide sequences into plants may vary depending on the type of plant or plant cell, that is, monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; McCabe et al. (1988) Biotechnology 6:923-926); and Lecl transformation (Int'l Patent Application Publication No. WO 00/28058). See also, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); each of which is incorporated herein by reference as if set forth in its entirety.

The cells that have been transformed may be grown into plants in accordance with conventional ways. See, e.g., McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure that expression of the desired phenotypic characteristic has been achieved.

The present invention may be used for transformation of any plant species, including, but not limited to, monocots and dicots. Examples of plants of interest include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatas), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp., Pisum spp.), and members of the genus Cucumis such as cucumber (C. sativus), cantaloupe (C. cantalupensis), and musk melon (C. melo). Ornamentals include azalea (Rhododendron spp.), hydrangea (Hydrangea macrophylla), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (e.g., corn, alfalfa, sunflower, Brassica, soybean, cotton, safflower, peanut, sorghum, wheat, millet, tobacco, etc.), more preferably corn and soybean plants, yet more preferably corn plants.

The methods of the invention can be used with other methods available in the art for enhancing disease resistance in plants. Similarly, the antimicrobial compositions described herein may be used alone or in combination with other nucleotide sequences, polypeptides, or agents to protect against plant diseases and pathogens. Although any one of a variety of second nucleotide sequences may be utilized, specific embodiments of the invention encompass those second nucleotide sequences that, when expressed in a plant, help to increase the resistance of a plant to pathogens.

The following examples are offered by way of illustration and not by way of limitation.

### EXPERIMENTAL

### Example 1

### Transformation and Regeneration of Transgenic Plants in Maize

Immature maize embryos from greenhouse donor plants are bombarded with a plasmid containing a CTTP operably linked to a defensin nucleotide sequence of the invention operably linked to a ubiquitin promoter and the selectable marker gene PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising a defensin nucleotide sequence of the invention operably linked to a ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for altered defense response defensin activity, insect resistance, nematode resistance, viral resistance, or fungal resistance.

Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂0 following adjustment to pH 5.8 with KOH); 2.0 g/l Gelrite (added after bringing to volume with D-I H₂0); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂0 following adjustment to pH 5.8 with KOH); 3.0 g/l Gelrite (added after bringing to volume with D-I H₂0); and 0.85 mg/l silver nitrate and 3.0 mg/l Bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂0) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂0 after adjusting to pH 5.6); 3.0 g/l Gelrite (added after bringing to volume with D-I H₂0); and 1.0 mg/l indoleacetic acid and 3.0 mg/l Bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂0), 0.1 g/1 myo-inositol, and 40.0 g/l sucrose (brought to volume with polished D-I H₂0 after adjusting pH to 5.6); and 6 g/l bacto-agar (added after bringing to volume with polished D-I H₂0), sterilized and cooled to 60° C.

### Example 2

### Agrobacterium-Mediated Transformation in Maize

For Agrobacterium-mediated transformation of maize with a CTTP peptide-defensin nucleotide sequence of the invention operably linked to a ubiquitin promoter, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the DNA construct containing the defensin nucleotide sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

### Example 3

### Soybean Embryo Transformation

Soybean embryos are bombarded with a plasmid containing the CTTP-defensin nucleotide sequence operably linked to a ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS 1000/HE instrument (helium retrofit) can be used for these transformations.

A selectable marker gene that can be used to facilitate soybean transformation is a transgene composed of the ³⁵S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the defensin nucleotide sequence operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to 7 days post bombardment, the liquid media may be exchanged with fresh media, and 11 to 12 days post-bombardment with fresh media containing 50 mg/ml hygromycin. This selective media can be refreshed weekly. Seven to 8 weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 4

### Sunflower Meristem Tissue Transformation

Sunflower meristem tissues are transformed with an expression cassette containing the CTTP-defensin sequence operably linked to a ubiquitin promoter as follows (see also EP Patent Application No. EP 0 486233, incorporated herein by reference as if set forth in its entirety, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al. (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant. 15:473-497), Shepard's vitamin additions (Shepard (1980) in Emergent Techniques for the Genetic Improvement of Crops University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6, and 8 g/l Phytagar.

The explants are subjected to microprojectile bombardment prior to Agrobacterium treatment (Bidney et al. (1992) Plant Mol. Biol. 18:301-313). Thirty to 40 explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the defensin gene operably linked to the ubiquitin promoter is introduced into Agrobacterium strain EHA105 via freeze-thawing as described by Holsters et al. (1978) Mol. Gen. Genet. 163:181-187. This plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for defensin-like activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by defensin-like activity analysis of leaf extracts while transgenic seeds harvested from NPTII-positive T₀ plants are identified by defensin-like activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it are dropped on the center of the surface of macrocarrier. Each plate is bombarded twice with 650 PSI rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/1 kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD₆₀₀. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for defensin-like activity using assays known in the art. After positive (i.e., for defensin expression) explants are identified, those shoots that fail to exhibit defensin-like activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate defensin-like protein activity assay. At this time, the positive shoots recovered from a single node will generally have been enriched in the transgenic sector detected in the initial assay prior to nodal culture.

Recovered shoots positive for defensin-like activity expression are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

### Example 5

### Assaying Defensin-Like Activity

The polypeptides described herein may be produced using any number of methods known to those skilled in the art. Such methods include, but are not limited to, expression in bacteria, eukaryotic cell cultures, in planta, and viral expression systems in suitably infected organisms or cell lines. The instant polypeptides may be expressed either as full-length polypeptides, mature forms, or as fusion proteins by covalent attachment to a variety of enzymes, proteins, or affinity tags. Common fusion protein partners include, but are not limited to, glutathione-S-transferase, thioredoxin, maltose binding protein, hexahistidine polypeptides, and chitin binding protein. The fusion proteins may be engineered with a protease recognition site at the fusion point so that fusion partners can be separated by protease digestion to yield intact mature peptides. Examples of such proteases include, but are not limited to, thrombin, enterokinase, and factor Xa. Indeed, any protease which specifically cleaves the peptide connecting the fusion protein and polypeptide of the invention can be used.

Purification of the polypeptides of the invention may utilize any number of separation technologies known to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography, and affinity chromatography. When the polypeptides of the invention are expressed as fusion proteins, the purification protocol may include the use of an affinity resin specific for the fusion protein partner or for the polypeptide of interest. Additional suitable affinity resins may be synthesized by linking the appropriate ligands to a suitable resin such as Sepharose-4B.

Crude, partially purified, or purified polypeptides of the invention, either alone or as a fusion protein, may be utilized in assays to verify expression levels of functional plant defensins in host cells and transgenic plants. Assays may be conducted under well known experimental conditions which permit optimal enzymatic activity. See, e.g., assays for plant defensin activities presented by Thevissen, K et al. (1996) J. Biol. Chem. 271:15018-15025 and Int'l Patent Application Publication No. WO 00/68405, each of which is incorporated herein by reference as if set forth in its entirety.

### Example 6

### Bioassay Testing the Pesticidal Activity of Polypeptides Against Southern Corn Rootworm (SCRW) and Western Corn Rootworm (WCRW)

Bio-Sery diet (catalog number F9800B, from: BIOSERV, Entomology Division, One 8^{th} Street, Suite 1, Frenchtown, N.J. 08825) is dispensed in 128-well CD International Bioassay trays (catalog number BIO-BA-128 from CD International, Pitman, N.J. 08071).

Protein samples are applied topically to the diet surface. Enough sample material is supplied to provide for replicate observations per sample. The trays are allowed to dry. Rootworms are dispensed into the wells of the bioassay trays. A lid (catalog number BIO-CV-16, CD International, Pitman, N.J., 08071) is placed on each tray, and the trays are placed in an incubator at 26° C. for 4 to 7 days.

For the evaluation of pesticidal activity against SCRW and WCRW, insects are exposed to a solution comprising either buffer (50 mM carbonate buffer (pH 10)) or a solution of protein sample at selected doses, for example, 50 or 5.0 μg/cm².

The bioassays are then scored by counting “live” versus “dead” larvae. Mortality is calculated as a percentage of dead larvae out of the total number of larvae tested.

### Example 7

### Bioassay Testing Pesticidal Activity of Polypeptides against the Colorado Potato Beetle (Leptinotarsa decemlineata)

Briefly, bioassay parameters are as follows: Bio-Sery diet (catalog number F9800B, from: BIOSERV, Entomology Division, One 8th Street, Suite 1, Frenchtown, N.J. 08825) is dispensed in a 96 well microtiter plate (catalog number 353918, Becton Dickinson, Franklin Lakes, N.J.) having a surface area of 0.33 cm². Protein samples of the invention are applied topically to the diet surface. Enough sample material is supplied to provide for 8 observations/sample. After the samples dry, 1 Colorado potato beetle neonate is added to each well providing for a total of 8 larvae/sample. A Mylar® lid (Clear Lam Packaging, Inc., 1950 Pratt Blvd., Elk Grove Village, Ill.) is affixed to each tray. Bioassay trays are placed in an incubator at 25° C. The test is scored for mortality on the 7th day following live infesting.

### Example 8

### Bioassay Testing Pesticidal Activity of Polypeptides against Lepidopterans

Neonate larvae are reared according to standard protocols, such as those published by Czapla and Lang (1990) J. Economic Entomology 83:2480-2485. Test compounds are either applied topically to the diet or incorporated into the larvae diet (see, id.). The larvae diet is dispensed to bioassay trays. One larva is applied per well of the bioassay tray. Weight and mortality are recorded 7 days following the start of the test.

### Example 9

### Homopteran Membrane Feeding Bioassay for Screening Proteins

This assay can be used for a variety of homopterans. The assay involves trapping the sample protein between two layers of maximally stretched parafilm that act as a sachet on top of a small vessel containing the insect of choice.

The assay is prepared as follows. 1 cm diameter polystyrene tubing is cut into 15 mm lengths. One end of the tube is then capped with a fine mesh screen. Five insects are then added to the chamber after which the first layer of parafilm is stretched over the remaining open end. 25 μl of sample (polypeptide in a 5% sucrose solution containing McCormick green food coloring) is then placed on top of the stretched parafilm. A second layer of parafilm is then stretched by hand and placed over the sample. The sample is spread between the two layers of parafilm to make a continuous sachet on which the insects feed. The sachet is then covered tightly with saran wrap to prevent evaporation and produce a slightly pressurized sample. The assay tubes are monitored for insect reproduction and death on a 24 hour basis and compared to the 5% sucrose control.

### Example 10

### Soybean Cyst Nematode Bioassay of Transgenic T0 Events

Soybean Cyst Nematodes (SCN) are used to infest transgenic T0 soybean plants in soil. SCN egg inoculum is acquired by harvesting cysts from plants infested 4-6 weeks earlier. Briefly, the soil is rinsed from the roots and passed through nested 20 mesh and 60 mesh screens. The material retained by the 20 mesh screen is discarded but the material retained by the 60 mesh screen is washed thoroughly and the creamy white cysts are recovered (older brown cysts are ignored). Similarly, the plant's root system is scrubbed against the 20 mesh screen nested over the 60 mesh screen. Cysts are harvested from the debris on the 60 mesh screen. Eggs are released from the cysts by means of a Dounce homogenizer in the presence of 0.5% Clorox for 2.5 minutes. Following this treatment the eggs are washed with sterile water from the homogenizer onto the surface of a 200 mesh screen. The eggs are then rinsed in water for an additional 5 minutes. Eggs are transferred to a 50 ml conical tube and counted. The eggs are diluted to 5000 eggs/ml. Plants grown in 15 cm conical tubes are inoculated with about 5000 eggs. Plants are maintained in a 26° C. growth chamber with 12:12 light:dark cycle for 1 month prior to harvest and counting of cysts.

### Example 11

### Bioactivity of Polypeptides Against Fungal Pathogens

The proteins of the invention are suspended in dH₂O to a final concentration of about 4 μg/μl. 12 μg of purified protein is added to 200 μl of ½ strength potato dextrose broth (PDB) containing a spore suspension of the fungal pathogen to be tested. The spore suspension contains approximately 2500 spores/ml. This results in a stock solution with a starting concentration of 10 μM. A 0.5× dilution series for the protein sample to be tested from 10 μM through to about 0.05 μM is prepared by removing 100 μl of the 10 μM stock and adding it to 100 μl of spore suspension (2500 spores/ml), mixing thoroughly to achieve a 5 μM protein sample concentration, transferring 100 μl of the 5 μM suspension to a fresh 100 μl spore suspension etc., until about 0.005 μM is reached. Two replicates per pathogen are performed. The fungal assay plate is scored for inhibition of fungal growth after a 48-hour incubation at 28° C. Inhibition of fungal growth is defined as little to no spore germination without detectable hyphae growth.

### Example 12

### Sequence Analysis of Defensin ZM-PDF20 Revealed a Putative CTTP

Bioinformatic analysis revealed that ZM-PDF20 contains a putative CTTP comprising 22 amino acids (amino acid sequence set forth in SEQ ID NO:1; predicted nucleotide sequence set forth in SEQ ID NO:2). The CTTP sequence begins immediately downstream of the C-terminal cysteine residue of the ZM-PDF20 polypeptide. The ZM-PDF20 CTTP (SEQ ID NO:1) contains a net charge of −2 and 12 hydrophobic amino acids (i.e., 55% hydrophobic). Distribution of expressed sequence tags within proprietary databases for the cDNA encoding ZM-PDF20 (SEQ ID NO:8) indicated that the transcript accumulates to high levels in pericarp and apical meristem tissue, consistent with the accumulation previously described for protein storage vacuole-targeted defensins in reproductive tissues.

### Example 13

### Analysis of Targeting of GFP by the ZM-PDF20 CTTP (SEQ ID NO:1)

The ability of the ZM-PDF20 CTTP set forth in SEQ ID NO:1 (encoded by the nucleotide sequence of SEQ ID NO:2) to target heterologous proteins to vacuole-like structures within transgenic plants was assayed using Aequorea coerulescens GFP (AcGFP; Clontech) driven by the maize ubiquitin promoter supplemented with its 5′UTR and the first intron (referred to herein as “ZM-UBI PRO” and set forth in SEQ ID NO:3(472). The ZM-PDF20 CTTP nucleotide sequence (SEQ ID NO:2) was fused directly behind the AcGFP sequence, followed by the PINII termination sequence. Targeting to the secretory pathway was accomplished by fusing the nucleotide sequence that encodes the barley alpha amylase (BAA) signal peptide (nucleotide sequence set forth in SEQ ID NO:4); amino acid sequence set forth in SEQ ID NO:Sdirectly in front of the AcGFP sequence. Experimental controls included similar fusion sequences in which the phaseolin CTTP (SEQ ID NO:6) or the tobacco chitinase CTTP (SEQ ID NO:7) were used in place of the ZM-PDF20 CTTP sequence, as described above. Maize suspension cells were transiently transformed with Agrobacterium containing a plasmid with one of the plant expression cassettes described herein and further set forth below. Microscopic observations were made after six days, the last two of which included exposure of the cells to an antibiotic to slow Agrobacterium growth. Vacuoles appeared as small-to-medium sized round bodies within the cytoplasm.

Results of targeting to particular part of the cell observed with transient expression of the various expression constructs are set forth in Table 1. Apoplastic targeting occurred when AcGFP was expressed without a CTTP. Nucleocytoplasmic targeting of AcGFP was observed when it was expressed along with the BAA signal peptide but without a CTTP. Fusion of the phaseolin CTTP (SEQ ID NO:6), the tobacco chitanse CTTP (SEQ ID NO:7), or the ZM-PDF20 CTTP (SEQ ID NO:1) to the AcGFP sequence resulted in vacuolar targeting of the marker protein in the plant cells. Previous studies have demonstrated that the phaseolin CTTP and the tobacco chitanse CTTP target proteins to plant vacuoles.

TABLE 1
Transient analysis of AcGFP targeting in maize suspension cells.

| Expression cassette | Observed targeting |
|---|---|
| UBI PRO:AcGFP:PINII TERM | Apoplastic |
| UBI PRO:BAA SS:AcGFP:PINII TERM | nucleocytoplasmic |
| UBI PRO:BAA SS:AcGFP:PHASEOLIN | Vacuolar |
| CTTP:PINII TERM | |
| UBI PRO:BAA SS:AcGFP:TOBACCO | Vacuolar |
| CHITINASE CTTP:PINII TERM | |
| UBI PRO:BAA SS:AcGFP:ZM-PDF20 | Vacuolar |
| CTTP:PINII TERM | |

### Example 14

### Transient Analysis of Targeting of Anti-Fungal Peptides by ZM-PDF20 CTTP

The ability of ZM-PDF20 CTTP to target potentially agronomically important proteins was assayed in the transient system described in Example 2. Constructs were made as in Example 2 in which defensin expression was driven by the UBI PRO (SEQ ID 473), and the ZM-PDF20 CTTP (SEQ ID NO:1) was fused directly behind either a plant defensin or a fungal defensin. Processing of the CTTP to produce a mature peptide was the criteria for targeting. Processing was assayed both by western blot and LC-MS analysis. Apoplastic targeting was assayed by measuring transgene accumulation in the suspension cell culture media. Controls included targeting of the same peptides using either the phaseolin CTTP (SEQ ID NO:6) or the tobacco chitinase CTTP (SEQ ID NO:7). Positive LC-MS detection, unless otherwise noted, indicates detection of completely processed mature peptide in a particular fraction (Table 2).

TABLE 2
Transient analysis of defensin targeting in maize suspension cells

| | Accumu- | | | |
|---|---|---|---|---|
| | lation | | | |
| | in cells | Accumulation | | |
| | (Western | in culture | LC/MS | LC/MS |
| Defensin +/− CTTP | blot) | (Western blot) | (cells) | (medium) |
| PP-PDF1 (7A4) | trace | +++ | + | +++ |
| PP-PDF1 | ++ | ++++ | ++ | +++ |
| (7A4):PHASEOLIN | | | | |
| CTTP | | | | |
| PP-PDF1 (7A4):ZM- | ++++ | trace | ++++* | +* |
| PDF20 CTTP | | | | |
| LE-DFN56 | no | no | nd | nd |
| LE- | + | + | nd | nd |
| DFN56:PHASEOLIN | | | | |
| CTTP | | | | |
| LE-DFN56:ZM- | ++++ | ++++ | +++* | ++* |
| PDF20 CTTP | | | | |
| LB-05197/8- | +++ | +++ | − | − |
| 1:TOBACCO | | | | |
| CHITINASE CTTP | | | | |
| LB-05197/8-1:ZM- | ++ | ++++ | − | − |
| PDF20 CTTP | | | | |
| LB-9827:ZM-PDF20 | nd | nd | +++ | nd |
| CTTP | | | | |
| LB- | no | no | nd | nd |
| 5220:PHASEOLIN | | | | |
| CTTP | | | | |
| LB-5220:TOBACCO | no | no | nd | nd |
| CHITINASE CTTP | | | | |
| LB-5220:ZM-PDF20 | no | no | nd | nd |
| CTTP | | | | |

*in addition to smaller amounts of species with incompletely processed C temini.

Western Methods

2 leaf punches/tube were lyophilized, pulverized, resuspended in 150 μl LDS-sample treatment buffer, 1% B-ME, and centrifuged. 75 μl of supernatant from 2 plants/event were pooled and then precipitated in 800 μl acetone overnight. The pellet was washed carefully with acetone and then resuspended in 100 μl LDS-sample treatment buffer, 1% B-ME, heated, and centrifuged. 25 μl of supernatant were loaded per lane.

Primary pAb from rabbits were used at 1:1000; and secondary goat anti-rabbit HRP-IgG at were used at 1:2000. ECL detection (Amersham™ ECL Western Blotting Detection Reagent) was performed for 15 sec followed by X-ray film-based detection. Stalk samples=1 core, approx. 1 cm×2.5 mm. BMS protein extraction and analysis similar, except that extraction buffer was as follows: 84 mM citric acid, 32 mM Na₂HPO₄, pH 2.8.

LC-MS (Liquid Chromatography/Mass spec)

30 mg dry weight BMS powder was suspended in 1% formic acid, sonicated 5 min, centrifuged at 14 K rpm, 15 min. Supernatant run through conditioned Oasis™ HLB (200 mg), washed with 5% AcN, 0.1% TFA, eluted with 5 mL 95% AcN, 0.1% TFA, dried, resuspended in 100 μL HPLC solvent A & filtered. Conditioning of the Oasis HLB was as follows: 5 ml MeOH, followed by 5 ml H₂O, then 5 ml 5% AcN, 0.1% TFA.

20 μl injections were made onto a Agilent Zorbax 300SB C8 3.5 μm 1.0 mm×150 mm column with Cap LCMS system.

Solvent A: 0.1% TFA+2.5% AcN (A), 97.5% AcN (B).

(AcN=acetonitrile; TFA=trifluoroacetic acid; MeOH=methanol; Oasis™ HLB=solid phase extraction columns with hydrophilic/lipophilic balance from Waters).

### Example 15

### Targeting of Anti-Fungal Peptides in Stably Transformed Maize by ZM-PDF20 CTTP

Accumulation of PP-PDF1 (7A4) and three other, closely related defensins was assayed in stably transformed maize at the T0 stage in leaf and stalk tissue using westerns. Generally, transgene product co-migrated with mature defensin standard, indicating appropriate processing. More transgene product accumulation was also generally observed in these experiments as compared to similar experiments in which defensin accumulation was targeted to the apoplast.

### Example 16

### Targeting of an Anti-Fungal Peptide in Stably Transformed Soy by ZM-PDF20 CTTP

The ability of the ZM-PDF20 CTTP (SEQ ID NO:1) to target heterologous proteins in soy was shown in a stably-transformed hairy root system, the method of Odell et al. (U.S. Pat. No. 7,196,247); and Subramanian et al. (2004) Plant Mol. Biol. 54: 623-639. The ZM-PDF20 CTTP was fused directly behind under the direction of a strong constitutive promoter. Expression of transgene produce in roots from six independent events was analyzed with quantitative western blots. Comparisons were made to accumulation of VP265 targeted using the proconcanavalin A CTTP (proCONA CTTP) of jack bean. VP265 co-migrated with mature defensin standard (not shown), indicating appropriate processing. These experiments show use of the ZM-PDF20 CTTP with yet another, plant-derived defensin in a heterologous dicot system to be beneficial, resulting in on average more accumulation of transgene product than with a previously described CTTP (Table 3). Overall, the average over 6 events was 7.46 ng for BAA SS:VP265-proCONA CTTP compared to 9.36 ng for BAA SS:VP265::ZMPDF20 CTTP.

TABLE 3
Accumulation of VP-265 in soy hairy roots
when targeted using the ZM-PDF20 CTTP

| | Blot 1 | | | Blot 2 | | |
|---|---|---|---|---|---|---|
| | | Avg. | | | Avg. | |
| Construct | Event | ng | Mean | Event | ng | Mean |
| BAA SS: VP265- | 11 | 5.42 | 5.68 | 26 | 9.14 | 9.24 |
| proCONA CTTP | 14 | 5.63 | | 27 | 9.47 | |
| | 15 | 5.98 | | 30 | 9.13 | |
| BAA | 33 | 18.59 | 12.55 | 6 | 7.73 | 6.16 |
| SS: VP265::ZM- | 53 | 7.02 | | 9 | 3.67 | |
| PDF20 CTTP | 54 | 12.05 | | 11 | 7.09 | |

All publications, patents and patent applications mentioned in the specification are indicative of the level of those skilled in the art to which this invention pertains. All publications, patents and patent applications are herein incorporated by reference to the same extent as if each individual publication, patent, or patent application was specifically and individually indicated to be incorporated by reference.

Although the foregoing invention has been described in some detail by way of illustration and example, for purposes of clarity of understanding, it will be obvious that certain changes and modifications may be practiced within the scope of the appended claims.

That which is claimed:

1. An isolated nucleic acid molecule comprising a nucleotide sequence selected from the group consisting of:
a) the nucleotide sequence of SEQ ID NO:2;
b) a nucleotide sequence having at least 90% sequence identity to SEQ ID NO:2, wherein the nucleotide sequence encodes a polypeptide, that when fused to a heterologous polypeptide of interest and expressed in a plant, increases accumulation of the heterologous polypeptide of interest to a vacuole of the plant;
c) a nucleotide sequence having at least 95% sequence identity to SEQ ID NO:2, wherein the nucleotide sequence encodes a peptide, that when fused to a heterologous polypeptide of interest and expressed in a plant, increases accumulation of the heterologous polypeptide of interest to a vacuole of the plant;
d) a nucleotide sequence that encodes the amino acid sequence of SEQ ID NO:1;
e) a nucleotide sequence that encodes an amino acid sequence having at least 90% sequence identity to SEQ ID NO:1, that when fused to a heterologous polypeptide of interest and expressed in a plant, increases accumulation of the heterologous polypeptide of interest to a vacuole of the plant; and
f) a nucleotide sequence encoding an amino acid sequence of a polypeptide having at least 95% sequence identity to SEQ ID NO:1, that when fused to a heterologous polypeptide of interest and expressed in a plant, increases accumulation of the heterologous polypeptide of interest to a vacuole of the plant.

2. A polypeptide encoded by the nucleic acid molecule of claim 1.

3. An expression cassette comprising a promoter that drives expression in a plant or plant cell operably linked to a polynucleotide that encodes a heterologous polypeptide of interest operably linked to a nucleic acid molecule of claim 1.

4. The expression cassette of claim 3 further comprising an operably linked polynucleotide encoding a signal peptide wherein the polynucleotide encoding the signal peptide comprises the nucleotide sequence of SEQ ID NO:4 or SEQ ID NO:5.

5. A transformed plant comprising the expression cassette of claim 3.

6. The plant of claim 5, wherein the plant is a monocot.

7. The plant of claim 6, wherein the monocot is maize, wheat, rice, barley, sorghum, or rye.

8. The plant of claim 5, wherein the plant is a dicot.

9. The plant of claim 8, wherein the dicot is soybean, Brassica, sunflower, cotton, or alfalfa.

10. A transformed seed of the plant of claim 5

11. A method for inducing plant pathogen resistance in a plant, said method comprising introducing into a plant at least one expression cassette according to claim 5.

12. An antipathogenic composition comprising a carrier and at least one polypeptide in accordance with claim 1.

13. A method for protecting a plant from a plant pathogen comprising applying the composition of claim 12 to the environment of a plant pathogen.

14. A method for increasing the accumulation of a polypeptide of interest in a vacuoles of a plant cell, said method comprising introducing into said plant cell a nucleic acid construct comprising a C-terminal targeting peptide (CTTP) having the amino acid sequence set forth in SEQ ID NO:1 operably linked to a nucleotide sequence encoding said polypeptide of interest.

15. The method of claim 14, wherein said polypeptide of interest is a defensin.

16. The method of claim 14, wherein said polypeptide of interest is a delta-endotoxin.

17. The method of claim 14, wherein said polypeptide of interest is a cry1Ab toxin.
